(19) United States
(12) Patent Application Publication
(10) Pub. No.: US 2002/0107404 A1
(43) Pub. Date: Aug. 8, 2002

(54) Sulfur-containing indirubin derivatives, their production and use

(75) Inventors: Olaf Prien, Berlin (DE); Andreas Steinmeyer, Berlin (DE); Gerhard Siemeister, Berlin (DE); Rolf Jautelat, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 09/983,548

(22) Filed: Oct. 24, 2001

(30) Foreign Application Priority Data
Oct. 24, 2000 (DE) 10053474.0-44

(51) Int. Cl. C07F 9/547; C 07D 4 3/04; C07D 491/02

(52) U.S. Cl. 548/414; 548/457; 548/431

(57) ABSTRACT

Sulfanyl-indirubin derivatives, their production and their intermediate products for production, as well as their use as medications for treating cancer, such as solid tumors and leukemia; auto-immune diseases, such as psoriasis, alopecia and multiple sclerosis, chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis, B and C, and HIV diseases, are described.

This invention relates to sulfur-containing indirubin derivatives, their production and their use as medications for treating various diseases.

It is known from traditional Chinese medicine that indirubin and several indirubin derivatives are effective against certain forms of cancer. In addition to antineoplastic actions, indirubin-3′-oxime-methyl ether and indirubin-3′-oxime-ethyl ether also show an in vitro inhibiting action on various leukemia cell lines of patients with acute lymphatic, acute myeloid and chronic granulocytic leukemia (Li et al., 1996, Bull. Chem. Soc. Japan, 69, 1621-1627 and Tian et al., 1995, Chemical Research in Chinese Universities, 11, 75-78).

As early as 1913, a patent on the production of alkyl ethers of indirubin oximes was granted by the German Imperial Patent Office (No. 282278).

The synthesis of selected indirubin derivatives, as well as their property as active active ingredients for treating cancer, thus for example as a preparation of the natural cocktail “Dang Gui Lu Hui Wan,” is described in Chinese J. Intern. Med. 15, 86-88, (1979).

Basic works for the synthesis of indirubin and indirubin derivatives are described in G. A. Russell, G. Kaupp, J. Am. Chem. Soc. 1969, 91, 3851-3859.

In addition, a pharmacological action of several indirubin derivatives is described in WO 99/62503.

Based on the advantageous properties of the compound class, a great need for more selective and especially more effective indirubin derivatives for treating various diseases continues to exist. This includes, for example, cancer, such as solid tumors and leukemia; auto-immune diseases, such as psoriasis, alopecia and multiple sclerosis, chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases.

It has now been found that sulfur-containing indirubin derivatives of general formula I,

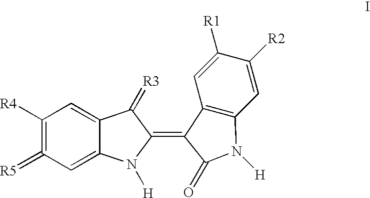

in which

R¹ and R² stand for hydrogen, halogen, hydroxy, nitroso, nitro, C₁-C₁₀ alkoxy optionally interrupted by one or more oxygen atoms; C₁-C₁₈ alkyl optionally substituted in one or more places with halogen, hydroxy and/or amino; aryl or heteroaryl optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl optionally substituted in one or more places with halogen, C₁-C₆ alkyl, hydroxy, amino and/or C₁-C₆ alkoxy and optionally containing one or more heteroatoms; or hydroxylamino, phosphate, phosphonate, sulfate, sulfonate, sulfonamide optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,

R³ stands for oxygen, sulfur, selenium, tellurium or the group ═NOR⁷ or ═NR⁹,

R⁴ and R⁵ stand for hydrogen, halogen, hydroxy, nitroso, nitro, C₁-C₁₀ alkoxy optionally interrupted by one or more oxygen atoms; C₁-C₁₈ alkyl optionally substituted in one or more places with halogen, hydroxy and/or amino; aryl, benzyl, benzyloxy or heteroaryl optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,

and

R⁶ stands for hydrogen, C₁-C₁₈ alkyl that is substituted in one or more places with halogen, hydroxy and/or amino; aryl, heteroaryl or C₃-C₈ cycloalkyl that is optionally substituted in one or more places with halogen, hydroxy, amino, C₁-C₆ alkyl and/or C₁-C₆ alkoxy,

or

R¹ and R²

or

R⁴ and R⁵, independently of one another, form a ring with 1 to 4 —CH₂ groups, which, independently of one another, optionally are substituted in one or two places with halogen, hydroxy, nitroso, nitro, C₁-C₁₀ alkoxy; C₁-C₁₈ alkyl that is optionally substituted in one or more places with halogen, hydroxy and/or amino; aryl or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —NHCO—C₁-C₆ alkyl group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,

R⁷ stands for hydrogen, C₁-C₁₈ alkyl, C₂-C₁₈ alkenyl, C₃-C₈ cycloalkyl or C₃-C₈ cycloalkenyl that is optionally interrupted by one or more oxygen atoms, or aryl or heteroaryl that is optionally substituted with hydroxy, halogen and/or amino,

R⁸ stands for aryl that is optionally substituted by one or more carboxyl, phosphoryl or sulfonate groups,

R⁹ stands for hydrogen, C₁-C₁₈ alkyl that is optionally substituted in one or more places with carboxy, phosphoryl or sulfonate groups, or for an aryl group with one or more heteroatoms that is optionally substituted with aralkyl or sulfonate,

R¹⁰ and R¹¹ are the same or different and mean hydrogen or C₁-C₁₈ alkyl, aryl, heteroaryl or acyl that is optionally substituted with hydroxy and/or amino; or C₁-C₁₈ alkyl, C₃-C₈ cycloalkyl or C₃-C₈ cycloalkenyl that is optionally interrupted by one or more oxygen atoms,

or

R¹⁰ and R¹¹ together with the nitrogen atom of the amino group forms a C₃-C₈ cycloalkyl, which can contain one or more additional heteroatoms,

M stands for hydrogen, C₁-C₁₈ alkyl that is optionally substituted by one or more hydroxy groups and/or amino groups, or aryl or heteroaryl that is optionally substituted in one or more places with halogen, C₁-C₆ alkyl or C₁-C₆ alkoxy,

n is 0, 1 or 2

and

at least one of radicals R¹, R², R⁴, and R⁵ is substituted with an —S(O)ₙR⁶ group, as well as optical isomers and salts thereof, show a surprising and, moreover, significantly better action on the isolated enzyme and on the cell compared to the known indirubin derivatives.

Alkyl is defined in each case as a straight-chain or branched alkyl radical, such as, for example, methyl, ethyl, propyl, isopropyl, butyl, isobutyl, sec-butyl, tert-butyl, pentyl, hexyl, heptyl, octyl, nonyl, decyl, undecyl, dodecyl, tridecyl, tetradecyl, pentadecyl, hexadecyl and octadecyl, whereby C₁-C₆ alkyl radicals are preferred.

Cycloalkyl is defined in each case as cyclopropyl, cyclobutyl, cyclopentyl, cyclohexyl, cycloheptyl and cyclooctyl.

Cycloalkenyl is defined in each case as cyclopropenyl, cyclobutenyl, cyclopentenyl, cyclohexenyl, cycloheptenyl and cyclooctenyl, whereby the linkage both to the double bond and to the single bonds can be carried out.

Alkyl, alkenyl, alkoxy, cycloalkyl and cycloalkenyl optionally can be interrupted by one or more oxygen atoms.

Halogen is defined in each case as fluorine, chlorine, bromine or iodine.

In each case, the-alkenyl substituents are straight-chain or branched and contain 2-18, preferably 2-10, especially 2-6 C atoms. For example, the following radicals can be mentioned: vinyl, propen-1-yl, propen-2-yl, but-1-en-1-yl, but-1-en-2-yl, but-2-en-1-yl, but-2-en-2-yl, 2-methyl-prop-2-en-1-yl, 2-methyl-prop-1-en-1-yl, but-1-en-3-yl, ethinyl, prop-1-in-1-yl, but-1-in-1-yl, but-2-in-1-yl, but-3-en-1-yl, allyl.

In each case, the aryl radical has 6-12 carbon atoms, such as, for example, naphthyl, biphenyl and especially phenyl.

In each case, the heteroaryl radical can be benzocondensed. For example, thiophene, furan, oxazole, thiazole, imidazole and benzo derivatives thereof can be mentioned as 5-ring heteroaromatic compounds, and pyridine, pyrimidine, triazine, quinoline, isoquinoline and benzo derivatives thereof can be mentioned as 6-ring heteroaromatic compounds.

If an acid group is included, the physiologically compatible salts of organic and inorganic bases are suitable as salts, such as, for example, the readily soluble alkali salts and alkaline-earth salts as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol.

If a basic group is included, the physiologically compatible salts of organic and inorganic acids are suitable, such as hydrochloric acid, sulfuric acid, phosphoric acid, citric acid, tartaric acid, i.a.

Especially effective are those compounds of general formula I, in which

R¹ stands for group —S(O)ₙR⁶,

R² stands for hydrogen,

R³ stands for oxygen or group ═NOR⁷,

R⁴ and R⁵ stand for hydrogen, halogen, hydroxy, nitroso, nitro, C₁-C₁₀ alkoxy that is optionally interrupted by one or more oxygen atoms; C₁-C₁₈ alkyl that is optionally substituted in one or more places with halogen, hydroxy and/or amino; aryl, benzyl, benzyloxy or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —NHCO-C₁-C₆ alkyl group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,

and

R⁶ stands for hydrogen or C₁-C₁₈ alkyl,

or

R¹ and R²

or

R⁴ and R⁵, independently of one another, form a ring with 1 to 4 —CH₂ groups, which, independently of one another, optionally are substituted in one or two places with halogen, hydroxy, nitroso, nitro, C₁-C₁₀ alkoxy; C₁-C₁₈ alkyl that is optionally substituted in one or more places with halogen, hydroxy and/or amino; aryl or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹group, —NHCO-C₁-C₆ alkyl group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,

R⁷ stands for hydrogen, C₂-C₁₈ alkenyl, or C₁-C₁₈ alkyl,

R⁸ stands for aryl that is optionally substituted by one or more carboxyl, phosphoryl or sulfonate groups,

R¹⁰ and R¹¹ are the same or different and mean hydrogen or C₁-C₁₈ alkyl, aryl, heteroaryl or acyl that is optionally substituted with hydroxy and/or amino; or C₁-C₁₈ alkyl, C₃-C₈ cycloalkyl or C₃-C₈ cycloalkenyl that is optionally interrupted by one or more oxygen atoms,

or

R¹⁰ or R¹¹ together with the nitrogen atom of the amino group forms a C₃-C₈ cycloalkyl, which can include one or more additional heteroatoms,

M stands for hydrogen, C₁-C₁₈ alkyl that is optionally substituted by one or more hydroxy groups and/or amino groups; or aryl or heteroaryl that is optionally substituted in one or more places with halogen, C₁-C₆ alkyl or C₁-C₆ alkoxy,

and

n is 0, 1 or 2, as well as optical isomers and salts thereof.

Especially effective are those compounds of general formula I, in which

R¹ stands for group —S(O)ₙR⁶,

R² stands for hydrogen,

R³ stands for oxygen or group ═NOR⁷,

R⁴ and R⁵ stand for hydrogen, halogen, hydroxy, nitroso, nitro, C₁-C₁₀ alkoxy that is optionally interrupted by one or more oxygen atoms; C₁-C₁₈ alkyl that is optionally substituted in one or more places with halogen, hydroxy and/or amino; aryl, benzyl, benzyloxy or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —NHCO-C₁-C₆ alkyl group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,

and

R⁶ stands for C₁-C₆ alkyl,

R⁷ stands for hydrogen or C₂-C₆ alkenyl,

R⁸ stands for aryl that is optionally substituted by one or more carboxyl, phosphoryl or sulfonate groups,

R¹⁰ and R¹¹ are the-same or different and mean hydrogen or C₁-C₁₈ alkyl, aryl, heteroaryl or acyl that is optionally substituted with hydroxy and/or amino; or C₁-C₁₈ alkyl, C₃-C₈ cycloalkyl or C₃-C₈ cycloalkenyl that is optionally interrupted by one or more oxygen atoms,

or

R¹⁰ and R¹¹ together with the nitrogen atom of the amino group forms a C₃-C₈ cycloalkyl, which can contain one or more additional heteratoms,

M stands for hydrogen, C₁-C₁₈ alkyl that is optionally substituted by one or more hydroxy groups and/or amino groups, or aryl or heteroaryl that is optionally substituted in one or more places with halogen, C₁-C₆ alkyl or C₁-C₆ alkoxy,

and

n is 0, 1 or 2, as well as optical isomers and salts thereof.

Those compounds of general formula I in which

R¹ stands for the group S(O)ₙR⁶,

R² stands for hydrogen,

R³ stands for oxygen or the group −NOR⁷

R⁴ stands for hydrogen, benzyloxy or for an —NHCOCH₃ group,

R⁵ stands for hydrogen,

R⁶ stands for C₁-C₆ alkyl,

R⁷ stands for hydrogen or C₂-C₆ alkenyl,

and

n is 0, 1 or 2, as well as optical isomers and salts thereof,

have proven especially effective.

The compounds according to the invention essentially inhibit cyclin-dependent kinases, upon which their action is based, for example, against cancer, such as solid tumors and leukemia; auto-immune diseases, such as psoriasis, alopecia and multiple sclerosis; chemotherapy agent-induced alopecia and mucositis; cardiovascular diseases, such as stenoses, arterioscleroses and restenoses; infectious diseases, such as, e.g., those caused by unicellular parasites, such as trypanosoma, toxoplasma or plasmodium, or those caused by fungi; nephrological diseases, such as, e.g., glomerulonephritis; chronic neurodegenerative diseases, such as Huntington's disease, amyotrophic lateral sclerosis, Parkinson's disease, AIDS dementia and Alzheimer's disease; acute neurodegenerative diseases, such as ischemias of the brain and neurotraumas; viral infections, such as, e.g., cytomegalic infections, herpes, hepatitis B and C, and HIV diseases.

The eukaryotic cell division cycle ensures the duplication of the genome and its distribution to the daughter cells by passing through a coordinated and regulated sequence of events. The cell cycle is divided into four successive phases: The GI phase represents the time before the DNA replication, in which the cell grows and is sensitive to external stimuli. In the S phase, the cell replicates its DNA, and in the G2 phase, preparations are made for entry into mitosis. In mitosis (M phase), the replicated DnA separates, and cell division is complete.

The cyclin-dependent kinases (CDKs), a family of Ser/Thr kinases, whose members require the binding of a cyclin (Cyc) as a regulatory subunit in order for them to activate, drive the cell through the cell cycle. Different CDK/Cyc pairs are active in the various phases of the cell cycle. CDK/Cyc pairs that are important to the basic function of the cell cycle are, for example, CDK4(6)/CycD, CDK2/CycE, CDK2/CycA, CDK1/CycA and CDK1/CycB. Some members of the CDK enzyme family have a regulatory function by influencing the activity of the above-mentioned cell cycle CDKs, while no specific function could be associated with other members of the CDK enzyme family. One of the latter, CDKS, is distinguished in that it has an atypical regulatory subunit (p35) that deviates from the cyclins, and its activity is highest in the brain.

The entry into the cell cycle and the passage through the “restriction points,” which marks the independence of a cell from further growth signals for the completion of the cell division that has begun, are controlled by the activity of the CDK4(6)/CycD and CDK2/CycE complexes. The essential substrate of these CDK complexes is the retinoblastoma protein (Rb), the product of the retinoblastoma tumor suppressor gene. Rb is a transcriptional co-repressor protein. In addition to other, still largely little understood mechanisms, Rb binds and inactivates transcription factors of the E2F type and forms transcriptional repressor complexes with histone-deacetylases (HDAC) (Zhang, H. S. et al. (2000). Exit from G1 and S Phase of the Cell Cycle is Regulated by Repressor Complexes Containing HDAC-Rb-hSWI/SNF and Rb-hSWI/SNF. Cell 101, 79-89). By the phosphorylation of Rb by CDKs, bonded E2F transcription factors are released and result in transcriptional activation of genes, whose products are required for the DNA synthesis and the progression through the S-phase. In addition, the Rb-phosphorylation brings about the breakdown of the Rb-HDAC complexes, by which additional genes are activated. The phosphorylation of Rb by CDK's is to be treated as equivalent to exceeding the “restriction points.” For the progression through the S-phase and its completion, the activity of the CDK2/CycE and CDK2/CycA complexes is necessary, e.g., the activity of the transcription factors of the E2F type is turned off by means of phosphorylation by CDK2/CycA as soon as the cells are entered into the S-phase. After replication of DNA is complete, the CDK1 in the complex with CycA or CycB controls the entry into and the passage through phases G2 and M (FIG. 1).

According to the extraordinary importance of the cell-division cycle, the passage through the cycle is strictly regulated and controlled. The enzymes that are necessary for the progression through the cycle must be activated at the correct time and are also turned off again as soon as the corresponding phase is passed. Corresponding control points (“checkpoints”) stop the progression through the cell cycle if DNA damage is detected, or the DNA replication or the creation of the spindle device is not yet completed.

The activity of the CDKs is controlled directly by various mechanisms, such as synthesis and degradation of cyclins, complexing of the CDKs with the corresponding cyclins, phosphorylation and dephosphorylation of regulatory Thr and Tyr radicals, and the binding of natural inhibitory proteins. While the amount of protein of the CDKs in a proliferating cell is relatively constant, the amount of the individual cyclins oscillates with the passage through the cycle. Thus, for example, the expression of CycD during the early G1 phase is stimulated by growth factors, and the expression of CycE is induced after the “restriction points” are exceeded by the activation of the transcription factors of the E2F type. The cyclins themselves are degraded by the ubiquitin-mediated proteolysis. Activating and inactivating phosphorylations regulate the activities of the CDKs, for example phosphorylate CDK-activating kinases (CAKs) Thr160/161 of the CDK1, while, by contrast, the families of Weel/Myt1 inactivate kinases CDK1 by phosphorylation of Thr14 and Tyr15. These inactivating phosphorylations can be destroyed in turn by cdc25 phosphatases. The regulation of the activity of the CDK/Cyc complexes by two families of natural CDK inhibitor proteins (CKIs), the protein products of the p21 gene family (p21, p27, p57) and the p16 gene family (p15, p16, p18, p19) is very significant. Members of the p21 family bind to cyclin complexes of CDKs 1,2,4,6, but inhibit only the complexes that contain CDK1 or CDK2. Members of the p16 family are specific inhibitors of the CDK4- and CDK6 complexes.

The plane of control point regulation lies above this complex direct regulation of the activity of the CDKs. Control points allow the cell to track the orderly sequence of the individual phases during the cell cycle. The most important control points lie at the transition from G1 to S and from G2 to M. The G1 control point ensures that the cell does not initiate any DNA synthesis unless it has proper nutrition, interacts correctly with other cells or the substrate, and its DNA is intact. The G2/M control point ensures the complete replication of DNA and the creation of the mitotic spindle before the cell enters into mitosis. The G1 control point is activated by the gene product of the p53 tumor suppressor gene. p53 is activated after detection of changes in metabolism or the genomic integrity of the cell and can trigger either a stopping of the cell cycle progression or apoptosis. In this case, the transcriptional activation of the expression of the CDK inhibitor protein p21 by p53 plays a decisive role. A second branch of the G1 control point comprises the activation of the ATM and Chk1 kinases after DNA damage by UV light or ionizing radiation and finally the phosphorylation and the subsequent proteolytic degradation of the cdc25A phosphatase (Mailand, N. et al. (2000). Rapid Destruction of Human cdc25A in Response to DNA Damage. Science 288, 1425-1429). A shutdown of the cell cycle results from this, since the inhibitory phosphorylation of the CDKs is not removed. After the G2/M control point is activated by damage of the DNA, both mechanisms are involved in a similar way in stopping the progression through the-cell cycle.

The loss of the regulation of the cell cycle and the loss of function of the control points are characteristics of tumor cells. The CDK-Rb signal path is affected by mutations in over 90% of human tumor cells. These mutations, which finally result in inactivating phosphorylation of the RB, include the over-expression of D- and E-cyclins by gene amplification or chromosomal translocations, inactivating mutations or deletions of CDK inhibitors of the p16 type, as well as increased (p27) or reduced (CycD) protein degradation. The second group of genes, which are affected by mutations in tumor cells, codes for components of the control points. Thus p53, which is essential for the G1 and G2/M control points, is the most frequently mutated gene in human tumors (about 50%). In tumor cells that express p53 without mutation, it is often inactivated because of a greatly increased protein degradation. In a similar way, the genes of other proteins that are necessary for the function of the control points are affected by mutations, for example ATM (inactivating mutations) or cdc25 phosphatases (over-expression).

Convincing experimental data indicate that CDK2/Cyc complexes occupy a decisive position during the cell cycle progression: (1) Both dominant-negative forms of CDK2, such as the transcriptional repression of the CDK2 expression by anti-sense oligonucleotides, produce a stopping of the cell cycle progression. (2) The inactivation of the CycA gene in mice is lethal. (3) The disruption of the function of the CDK2/CycA complex in cells by means of cell-permeable peptides resulted in tumor cell-selective apoptosis (Chen, Y. N. P. et al. (1999). Selective Killing of Transformed Cells by Cyclin/Cyclin-Dependent Kinase 2 Antagonists. Proc. Natl. Acad. Sci. USA 96, 4325-4329).

Changes of the cell cycle control play a role not only in carcinoses. The cell cycle is activated by a number of viruses, both by transforming viruses as well as by non-transforming viruses, to make possible the reproduction of viruses in the host cell. The false entry into the cell cycle of normally post-mitotic cells is associated with various neurodegenerative diseases. The mechanisms of the cell cycle regulation, their changes in diseases and a number of approaches to develop inhibitors of the cell cycle progression and especially the CDKs were already described in a detailed summary in several publications (Sielecki, T. M. et al. (2000). Cyclin-Dependent Kinase Inhibitors: Useful Targets in Cell Cycle Regulation. J. Med. Chem. 43, 1-18; Fry, D. W. & Garrett, M. D. (2000). Inhibitors of Cyclin-Dependent Kinases as Therapeutic Agents for the Treatment of Cancer. Curr. Opin. Oncol. Endo. Metab. Invest. Drugs 2, 40-59; Rosiania, G. R. & Chang, Y. T. (2000). Targeting Hyperproliferative Disorders with Cyclin-Dependent Kinase Inhibitors. Exp. Opin. Ther. Patents 10, 215-230; Meijer L. et al. (1999). Properties and Potential Applications of Chemical Inhibitors of Cyclin-Dependent Kinases. Pharmacol. Ther. 82, 279-284; Senderowicz, A. M. & Sausville E. A. (2000). Preclinical and Clinical Development of Cyclin-Dependent Kinase Modulators. J. Natl. Cancer Inst. 92, 376-387).

To use the compounds according to the invention as pharmaceutical agents, the latter are brought into the form of a pharmaceutical preparation, which in addition to the active ingredient for enteral or parenteral administration contains suitable pharmaceutical, organic or inorganic inert support media, such as, for example, water, gelatin, gum arabic, lactose, starch, magnesium stearate, talc, vegetable oils, polyalkylene glycols, etc. The pharmaceutical preparations can be present in solid form, for example as tablets, coated tablets, suppositories, capsules or in liquid form, for example as solutions, suspensions, or emulsions. Moreover, they optionally contain adjuvants, such as preservatives, stabilizers, wetting agents or emulsifiers; salts for changing the osmotic pressure or buffers.

These pharmaceutical preparations are also subjects of this invention.

For parenteral administration, especially injection solutions or suspensions, especially aqueous solution of active compounds in polyhydroxyethoxylated castor oil are suitable.

As carrier systems, surface-active adjuvants such as salts of bile acids or animal or plant phospholipids, but also mixtures thereof as well as liposomes or their components, can also be used.

For oral administration, especially tablets, coated tablets or capsules with talc and/or hydrocarbon vehicles or binders, such as, for example, lactose, corn or potato starch, are suitable. The administration can also be carried out in liquid form, such as, for example, as a juice, to which optionally a sweetener is added.

Enteral, parenteral and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose to be administered once or divided into two or more daily doses.

Subjects of this invention also include the use of compounds of general formula I for the production of a pharmaceutical agent for treating cancer, auto-immune diseases, cardiovascular diseases, chemotherapy agent-induced alopecia and mucositis, infectious diseases, nephrological diseases, chronic and acute neurodegenerative diseases and viral infections, whereby cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B or C, and HIV diseases.

Subjects of this invention also include pharmaceutical agents for treating the above-cited diseases, which contain at least one compound according to general formula I, as well as pharmaceutical agents with suitable formulation substances and vehicles.

The compounds of general formula I according to the invention are, i.a., excellent inhibitors of the cyclin-dependent kinases, such as CDK1, CDK2, CDK3, CDK4, CDK5, CDK6, CDK7, CDK8 and CDK9, as well as the glycogen-synthase-kinase (GSK-3β).

The compounds of general formula I according to the invention can be produced by it being possible to distinguish a compound of general formula II

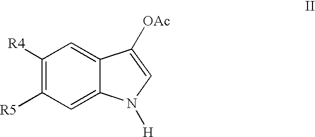

in which R⁴ and R⁵ have the meanings that are indicated in general formula I, with a compound of general formula III

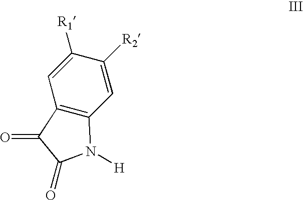

in which R¹′ and R²′ have the meanings that are indicated in general formula I under R¹ and R², and by being able to distinguish R¹′ and R²′ from R¹ and R² to the extent that the oxidation stage in the sulfur does not necessarily have to correspond to the final stage.

If the production of the starting compounds is not described, these compounds are known or can be produced analogously to known compounds or to processes that are described here. It is also possible to perform all reactions that are described here in parallel reactors or by means of combinatory operating procedures.

The isomer mixtures can be separated into the enantiomers or E/Z isomers according to commonly used methods, such as, for example, crystallization, chromatography or salt formation.

The production of the salts is carried out in the usual way by a solution of the compound of formula I being mixed with the equivalent amount of or excess base or acid, which optionally is in solution, and the precipitate being separated or the solution being worked up in the usual way.

The following examples explain the production of the compounds according to the invention.

The production of the compounds according to the invention is carried out essentially according to the following diagram:

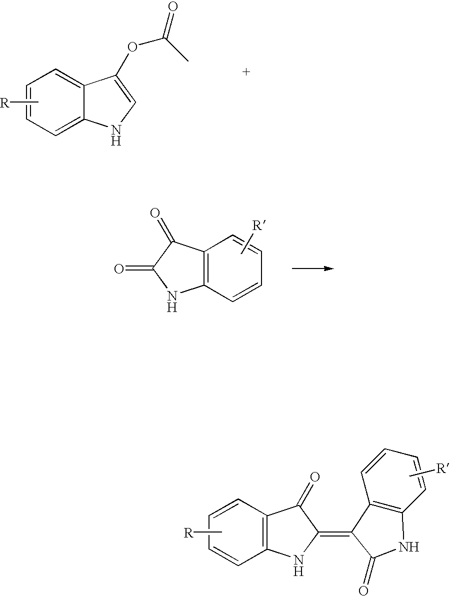

Hereinafter, R stands for R⁴ and R⁵, and R′ stands for R¹ and R² of general formula I.

Moreover, the introduction of substituents can also be carried out in a transformation that is downstream to the formation of indirubins.

The synthesis of indirubins can be carried out, for example, analogously to the reaction that is described in the literature by reaction of the corresponding isatin with an indoxyl acetate. To increase the yield and to minimize secondary reactions, the reaction should be carried out under a cover gas (e.g., argon, nitrogen). The general synthesis technique is described in the literature (G. A. Russell, G. Kaupp, J. Am. Chem. Soc. 1969, 91, 3851-3859).

The production of sulfoxides is carried out by reaction of the corresponding sulfanyl derivatives in the presence of an oxidizing agent. This can be, for example, mCPBa or H₂O₂, etc. The prior art is described extensively in the literature (cf. S. Uemura in Trost, Fleming Comprehensive Organic Synthesis, Volume 7, Pergamon Press, 1991, 757-787; M. Madesclaire, Tetrahedron, 4, 1986, 5459-5495; J. Drabowicz, M. Mikolajczyk Org. Prep. Proced. Int. 1982, 14, 45-89). As an alternative, synthesis of indirubin-sulfoxides can also be carried out by conversion of suitable starting materials (for example thio-isatins or thio-indoxyl acetates) into the corresponding sulfoxides and subsequent condensation into the indirubins. In the presence of suitable catalysts, the enantiomers can also be produced specifically (S. Uemura in Trost, Fleming Comprehensive Organic Synthesis, Volume 7, Pergamon Press, 1991, 777-787). The oxidation in presence of enzymes can also be implemented (cf. H. L. Holland, Chem. Rev. 1988, 88, 473-485).

The production of sulfones is carried out by reaction of the corresponding sulfanyl derivatives in the presence of an oxidizing agent; this can be, for example, mCPBA or H₂O₂, etc. The prior art is described extensively in the literature (cf. S. Uemura in Trost, Fleming Comprehensive Organic Synthesis, Volume 7, Pergamon Press, 1991, 757-787). As an alternative, synthesis of indirubin sulfoxides can also be carried out by conversion of suitable starting materials (for example thio-isatins or thio-indoxyl acetates) into the corresponding sulfoxides and subsequent condensation into the indirubins.

The synthesis of oximes is carried out by reaction of the corresponding carbonyl compound with hydroxylamine, analogously to the instructions that are known in the literature (C. Li et al., Bull. Chem. Soc. Jpn. (1996), 69, 1621-1627).

The production of oxime-ethers (see diagram) is carried out from the corresponding oximes by base-catalyzed etherification in the presence of an alkylating agent. As a base for this reaction, inorganic or organic bases can be used. As a solvent,

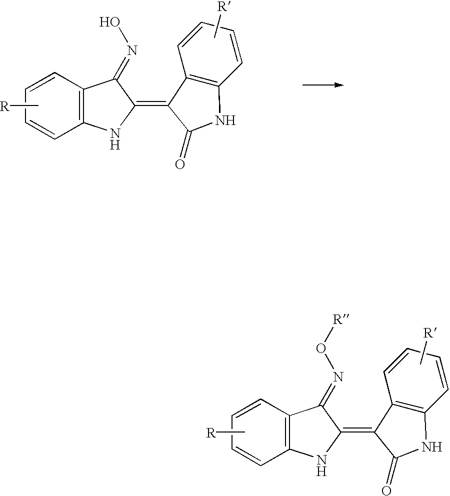

protic or aprotic polar media are used.

In the diagram, R has the meaning of R⁴ and R⁵, R″ has the meaning of R⁷, and R′ has the meaning of R¹ and R² of general formula I.

The following examples explain the production of starting compounds that are required for the synthesis of indirubin derivatives.

Production of Isatins

A-1) 5-Methylsulfanyl-1H-indole-2,3-diones

The synthesis of 5-methylsulfanyl-1H-indole-2,3-diones is carried out analogously to the instructions that are described in the literature (cf. K. Brand, E. V8lker, Arch. Pharm. 1934, 272, 257-268). Synthesis into isatins can also be carried out, however, by lithiation of the corresponding educts and subsequent condensation (cf. P. Hewawasam, N. A. Meanwell, Tetrahedron Lett. 1994, 35, 7303-7306) or by palladium-catalyzed reaction in the presence of carbon monoxide (cf. K. Smith, G. A. El-Hiti, A. C. Hawes, Synlett, 1999, 945-947).

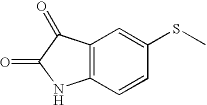

¹H NMR (DMSO-d₆): δ2.48 (s, 3H), 6.89 (d,. 1H, J=9 Hz), 7.42 (dd, 1H, J=3 Hz), 7.54 (dd, 1H), J=9 Hz, J=3 Hz), 11.05 (s, 1H).

MS (ESI): 193 (43) (M), 165 (100), 122 (80).

Melting point: 170° C. (decomposition)

A-2) 5-Methylsulfonyl-1H-indole-2,3-diones 193 mg (1.0 mmol) of 5-methylsulfanyl-1H-indole-2,3-diones were dissolved at room temperature in 12 ml of dichloromethane and mixed in portions with a total of 496 mg (2.3 mmol) of m-chloroperbenzoic acid. After the addition was completed, the reaction mixture was stirred for 24 hours at room temperature. For working-up, it was diluted with dichloromethane, and the organic phase was extracted twice with saturated sodium bicarbonate solution. The aqueous phase is vigorously acidified with hydrochloric acid and extracted again with ethyl acetate. The combined organic phases are washed with saturated sodium chloride solution and dried on sodium sulfate. After chromatographic purification on silica gel, 59 mg (26%) of the desired product was obtained.

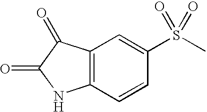

¹H NMR (DMSO-D₆): δ3.22 (s, 3H), 7.12 (d, 1H), 7.96 (d, 1H), 8.10 (dd, 1H), 11.47 (s, 1H).

MS (ESI): 225 (38) (M), 197 (100).

Melting point: 260-263° C.

B-1) Production of the Indoxylacetates The synthesis of indoxylacetates is carried out analogously to the process that is mentioned in the literature (Friedlaender Bruckner, Justus Liebigs Ann. Chem. 1912, 388). If the compounds were commercially available, the latter were used for synthesis without previous purification.

The following examples explain the production of the compounds according to the invention without limiting the scope of the claimed compounds to these examples.

### Example 1.0

5-Methylsulfanyl-indirubin

22 ml of methanol, p.a., is introduced into a flask under nitrogen. 467 mg (2.58 mmol) of indoxylacetate, 500 mg (2.58 mmol) of 5-methylsulfanyl-1H-indole-2,3-dione and 603 mg (5.69 mmol) of sodium carbonate are added in succession, and the reaction mixture is degassed for 20 minutes while nitrogen is passing through it. Then, the reaction mixture is stirred overnight at room temperature. After the reaction is completed, the crystals are separated by filtration and washed neutral with water. The residue is recrystallized from hot ethanol and dried in a vacuum. The desired product is isolated in the form of dark-violet crystals (503 mg, 63%). By crystallization of the mother liquor, another 13% (101 mg) of the desired compound could be obtained.

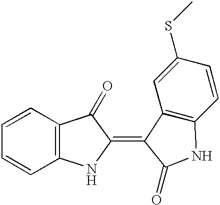

¹H-NMR (acetone-D₆): δ2.56 (s, 3H), 6.98 (d, 1H), J=8 Hz), 7.10 (dt, 1H, J=7 Hz), 7.28 (dd, 2H), 7.43 (dd, 1H), 7.63 (dt, 1H), 7.73 (dd, 1H), 8.97 (d, 1H), 9.89 (br s, 1H), 10.83 (br s, 1H).

MS % (ESI): 308 (100) (M+H)

Melting point: >210° C. (ethanol)

The following compounds are also produced by a similar method:

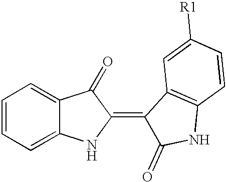

| Example No. | R¹ | Melting point [° C.] and MS |
|---|---|---|
| 1.1 | —SO₂CH₃ | >300 |
| | | El: M + 340(100%), |
| | | 262(68%), 233(71%) |

### Example 2.0

5-Methylsulfinyl-indirubin 200 mg (0.5 mmol) of 5-methylsulfanyl-indirubin is dissolved in 50 ml of dichloromethane at room temperature. The solution is slowly mixed with 125 mg (0.65 mmol) of m-chloroperbenzoic acid. After the addition is completed, it is allowed to stir for two more hours at room temperature, it is worked up in aqueous form with saturated sodium bicarbonate solution, and the organic phase is dried on sodium sulfate. The final chromatographic purification of the crude product on silica gel yields the desired product in the form of deep-violet crystals (75 mg, 36%).

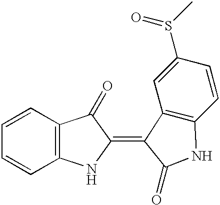

¹H-NMR (acetone-D₆): δ3.04 (s, 3H), 7.10 (dt, 1H), 7.16 (d, 1H), 7.48 (dd, 2H), 7.63 (m, 2H), 7.73 (dd, 1H), 9.18 (d, 1H), 10.92 (br s, 1H1), 11.02 (br s, 1H).

MS % (ESI): 325 (100) (M+H); 309 (57) (M−C).

Melting point: 284° C. (ethanol)

### Example 4.0

5-Methylsulfanyl-3′,-hydroyimino-indirubin 50 mg (0.16 mmol) of 5-methylsulfanyl-indirubin is dissolved in 1 ml of ethanol. 50 mg (0.72 mmol) of hydroxylammonium chloride and 200 mg (3.56 mmol) of solid potassium hydroxide are added in succession. The reaction mixture is refluxed. After one hour, the reaction mixture is cooled and mixed with water. The solid is filtered off, and the filtrate is acidified with acetic acid. The precipitate that is formed is filtered off and washed with water. The product is obtained in the form of red needles (10 mg, 31%).

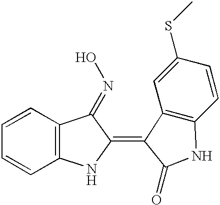

MS % (ESI): 324 (98) (M+H); 308 (100) (M−NOH).

Melting point: 156° C.

The following compounds are also produced by a similar method:

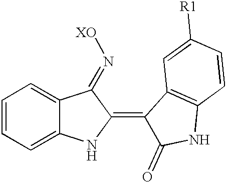

| | | | Melting point [° C.] |
|---|---|---|---|
| Example No. | X | R¹ | and MS |
| 4.1 | H | —S(O)CH₃ | 274 |
| 4.2 | CH₂═CH—CH_{2—} | —SCH₃ | El: M + 364(100%), |
| | | | 258(28%), 120(40%) |

### Example 5.0

5-Methysulfanyl-5′-N-acetylindirubin

121 mg (0.52 mmol) of the compound

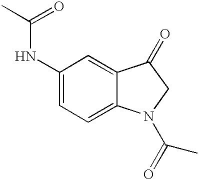

and 127 mg (0.5 mmol) of the compound

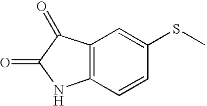

are suspended in 3 ml of glacial acetic acid, mixed with 0.1 ml of concentrated hydrochloric acid and then stirred under argon atmosphere for 5 hours at 90° C. After cooling, it is diluted with ethanol. Then, the crystals are suctioned off and dried in a vacuum.

Yield: 53% of theory of compound

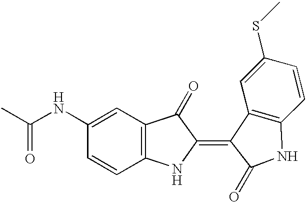

DMSO-d₆: 2.05 (3H, s), 6.87 (1H, d, J=8 Hz), 7.21 (1H, d d, J=8 Hz, 1 Hz), 7.33 (1H, d, J=8 Hz), 7.6 (1H, d d, J=8 Hz, 1 Hz), 8.0 (1H, d, J=1 Hz), 8.8 (1H, d, J=1 Hz), 10.05 (1H, s), 10.89 (1H, s), 10.98 (1H, s).

EI: M+365 (100%), 323 (40%), 280 (18%).

The following compound is also produced by a similar method:

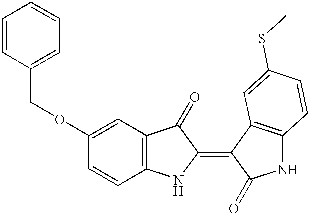

EI: M+414 (30%), 323 (100%), 91 (70%).

### DESCRIPTION OF THE FIGURE

FIG. 1 shows the simplified diagram of cell cycle regulation in vertebrates.

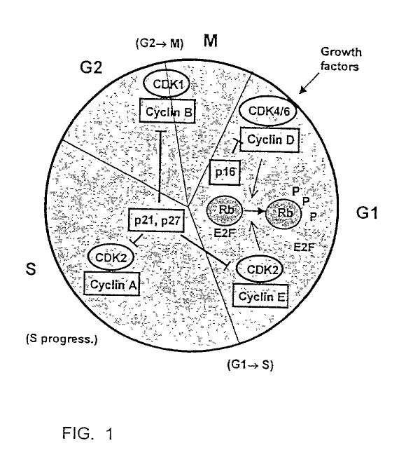

The following examples describe the biological action of the compounds according to the invention without limiting the invention to these examples.

### Example 1

CDK2/CycE Kinase Assay

Recombinant CDK2- and CycE-GST-fusion proteins, purified from baculovirus-infected insect cells (Sf9), were obtained by Dr. Dieter Marmé, Klinik für Tumorbiologie [Clinic for Tumor Biology], Freiburg. Histone IIIS, which was used as a kinase substrate, was purchased by the Sigma Company.

CDK2/CycE (50 ng/measuring point) was incubated for 15 minutes at 22° C. in the presence of various concentrations of test substances (0 μm, as well as within the range of 0.01-100 μm) in assay buffer [50 mmol of tris/HCl pH 8.0, 10 mmol of MgCl₂, 0.1 mmol of Na ortho-vanadate, 1.0 mmol of dithiothreitol, 0.5 μm of adenosine triphosphate (ATP), 10 μg/measuring point of histone IIIS, 0.2 μCi/measuring point of ³³P-gamma ATP, 0.05% NP40, 12.5% dimethyl sulfoxide]. The reaction was stopped by adding EDTA solution (250 mmol, pH 8.0, 14 μl/measuring point).

From each reaction batch, 10 μl was applied to P30 filter strips (Wallac Company), and non-incorporated ³³P-ATP was removed by subjecting the filter strips to three washing cycles for 10 minutes each in 0.5% phosphoric acid. After the filter strips were dried for one hour at 70° C., the filter strips were covered with scintillator strips (MeltiLex™ A, Wallac Company) and baked for one hour at 90° C. The amount of incorporated ³³p (substrate phosphorylation) was determined by scintillation measurement in a gamma-radiation measuring device (Wallac).

### Example 2

CDK1/CycB Kinase Assay

Recombinant CDK1- and CycB-GST fusion proteins, purified from baculovirus-infected insect cells (Sf9), were obtained by Dr. Dieter Marmé, Klinik für Tumorbiologie Freiburg. Histone IIIS, which was used as a kinase substrate, was purchased by the Sigma Company.

CDK1/CycB (50 ng/measuring point) was incubated for 15 minutes at 22° C. in the presence of various concentrations of test substances (0 μm, as well as within the range of 0.01-100 μm) in assay buffer [50 mmol of tris/HCl pH 8.0, 10 mmol of MgCl₂, 0.1 mmol of Na ortho-vanadate, 1.0 mmol of dithiothreitol, 0.5 μm of adenosine triphosphate (ATP), 10 μg/measuring point of histone IIIS, 0.2 μCi/measuring point of ³³P-gamma ATP, 0.05% NP40, 12.5% dimethyl sulfoxide]. The reaction was stopped by adding EDTA solution (250 mmol, pH 8.0, 14 μl/measuring point).

From each reaction batch, 10 μl was applied to P30 filter strips (Wallac Company), and non-incorporated ³³P-ATP was removed by three washing cycles of the filter strips for 10 minutes each in 0.5% phosphoric acid. After the filter strips were dried for 1 hour at 70° C., the filter strips were covered with scintillator strips (MeltiLex™ A, Wallac Company) and baked for one hour at 90° C. The amount of incorporated ³³p (substrate phosphorylation) was determined by scintillation measurement in a gamma-radiation measuring device (Wallac).

### Example 3

CDK4/CycD1 Kinase Assay

Recombinant CDK4- and CycD1-GST fusion proteins, purified from baculovirus-infected insect cells (Sf9), were obtained by Dr. Dieter Marmé, Klinik für Tumorbiologie Freiburg. The kinase substrate, a GST-fusion protein of the 20 kD C-terminal fragment of the Rb protein, was obtained by Dr. Dieter Marmé, Klinik für Tumorbiologie Freiburg.

CDK4/CycD1 (200 ng/measuring point) was incubated for 15 minutes at 22° C. in the presence of various concentrations of test substances (0 μm, as well as within the range of 0.01-100 μm) in assay buffer [50 mmol of tris/HCl pH 8.0, 10 mmol of MgCl₂, 0.1 mmol of Na ortho-vanadate, 1.0 mmol of dithiothreitol, 0.5 μm of adenosine triphosphate (ATP), 1 μg/measuring point of C-terminal Rb-GST-fusion protein, 0.2 μCi/measuring point of ³³P-gamma ATP, 0.05% NP40, 12.5% dimethyl sulfoxide]. The reaction was stopped by adding EDTA solution (250 mmol, pH 8.0, 14 μl/measuring point).

From each reaction batch, 10 μl was applied to P30 filter strips (Wallac Company), and non-incorporated ³³P-ATP was removed by three washing cycles of the filter strips for 10 minutes each in 0.5% phosphoric acid. After the filter strips were dried for 1 hour at 70° C., the filter strips were covered with scintillator strips (MeltiLex™ A, Wallac Company) and baked for one hour at 90° C. The amount of incorporated ³³p (substrate phosphorylation) was determined by scintillation measurement in a gamma-radiation measuring device (Wallac).

### Example 4

Proliferation Assay

Cultivated human tumor cells (as indicated) were flattened out at a density of 5000 cells/measuring point in a 96-well multititer plate in 200 μl of the corresponding growth medium. After 24 hours, the cells of one plate (zero-point plate) were colored with crystal violet (see below), while the medium of the other plates was replaced by fresh culture medium (200 μl), to which the test substances were added in various concentrations (0 μm, as well as in the range of 0.01-30 μm; the final concentration of the solvent dimethyl sulfoxide was 0.5%. The cells were incubated for 4 days in the presence of test substances. The cell proliferation was determined by coloring the cells with crystal violet: the cells were fixed by adding 20 μl/measuring point of a 11% glutaric aldehyde solution for 15 minutes at room temperature. After three washing cycles of the fixed cells with water, the plates were dried at room temperature. The cells were colored by adding 100 μl/measuring point of a 0.1% crystal violet solution (pH was set at 3 by adding acetic acid). After three washing cycles of the colored cells with water, the plates were dried at room temperature. The dye was dissolved by adding 100 μl/measuring point of a 10% acetic acid solution. The extinction was determined by photometry at a wavelength of 595 nm. The change of cell growth, in percent, was calculated by standardization of the measured values to the extinction values of the zero-point plate (=0%) and the extinction of the untreated (0 μm) cells (=100%).

The results of the tests are cited in the following tables.

| | CDK2 | MCF-7 |
|---|---|---|
| Example No. | IC₅₀ [μM] | IC₅₀ [μM] |
| 5.0 | 0.11 | 5 |
| 5.1 | 5 | — |

Proof of Superiority of the Compounds According to the Invention Compared to the Known Compounds

To prove the superiority of the compounds according to the invention compared to the known compounds, the compounds according to the invention were compared to structurally-similar known compounds both in the enzyme test and in the cell test. The result is cited in the following table:

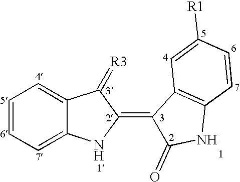

| | | | CDK2 | MCF-7 | H460 | HCT116 | DU145 |
|---|---|---|---|---|---|---|---|
| Example | | | IC₅₀ | IC₅₀ | IC₅₀ | IC₅₀ | IC₅₀ |
| No. | R¹ | R³ | [μM] | [μM] | [μM] | [μM] | [μM] |
| 1.0 | SMe | O | 0.3 | 0.5 | | | |
| 1.1 | SO₂Me | O | 0.07 | >10 | | | |
| 2.0 | SOMe | O | 0.08 | 1.5 | 4.0 | 2.5 | 6 |
| 4.1 | SOMe | NOH | 0.08 | 1.5 | 3.0 | 2.0 | 3.0 |
| 4.0 | SMe | NOH | 0.02 | 0.7 | 1.0 | 0.6 | 1.0 |
| Compound No. 11, page 14, | SO₃H | O | 0.3 | >10 | >10 | >10 | >10 |
| Table 2 of WO 99/62503 | | | | | | | |
| Compound No. 6, page 14, | Me | O | 3 | 1 | >10 | 10 | >10 |
| Table 2 of WO 99/62503 | | | | | | | |
| Compound No. 10, page 14, | H | NOH | 0.9 | 6 | | | |
| Table 2 of WO 99/62503 | | | | | | | |

Me = methyl

It can be seen from the table that both in the enzyme test and in the cell test, the compounds according to the invention have significantly higher activities in the enzyme and in the MCF-7 cells than the compounds that are known from the closest prior art (WO 99/62503). The compounds according to the invention are thus far superior to the known compounds. In this connection, it is to be noted that the substituent at R¹ is decisive to the superiority of the compounds according to the invention and the other substituents do not produce any significant change in the effectiveness of the basic compounds.

1. Sulfur-containing indirubin derivatives of general formula I 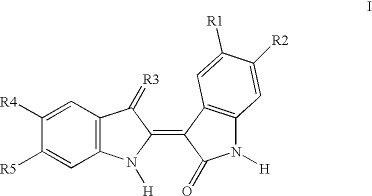
in which
R¹ and R² stand f or hydrogen, halogen, hydroxy, nitroso, nitro, C₁-C₁₀ alkoxy optionally interrupted by one or more oxygen atoms; C₁-C₁₈ alkyl optionally substituted in one or more places with halogen, hydroxy and/or amino; aryl or heteroaryl optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl optionally substituted in one or more places with halogen, C₁-C₆ alkyl, hydroxy, amino and/or C₁-C₆ alkoxy and optionally containing one or more heteroatoms; or hydroxylamino, phosphate, phosphonate, sulfate, sulfonate, sulfonamide optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,
R³ stands for oxygen, sulfur, selenium, tellurium or the group ═NOR⁷ or ═NR⁹,
R⁴ and R⁵ stand for hydrogen, halogen, hydroxy, nitroso, nitro, C₁-C₁₀ alkoxy optionally interrupted by one or more oxygen atoms; C₁-C₁₈ alkyl optionally substituted in one or more places with halogen, hydroxy and/or amino; aryl, benzyl, benzyloxy or heteroaryl optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —NHCO-C₁-C₆ alkyl group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,
and
R⁶ stands for hydrogen, C₁-C₁₈ alkyl that is substituted in one or more places with halogen, hydroxy and/or amino; aryl, heteroaryl or C₃-C₈ cycloalkyl that is optionally substituted in one or more places with halogen, hydroxy, amino, C₁-C₆ alkyl and/or C₁-C₆ alkoxy,
or
R¹ and R²
or
R⁴ and R⁵, independently of one another, form a ring with 1 to 4 —CH₂ groups, which, independently of one another, optionally are substituted in one or two places with halogen, hydroxy, nitroso, nitro, C₁-C₁₀ alkoxy; C₁-C₁₈ alkyl that is optionally substituted in one or more places with halogen, hydroxy and/or amino; aryl or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₆ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰ OR¹¹ group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,
R⁷ stands for hydrogen, C₁-C₁₈ alkyl, C₂-C₁₈ alkenyl, C₃-C₈ cycloalkyl or C₃-C₈ cycloalkenyl that is optionally interrupted by one or more oxygen atoms, or aryl or heteroaryl that is optionally substituted with hydroxy, halogen and/or amino,
R⁸ stands for aryl that is optionally substituted by one or more carboxyl, phosphoryl or sulfonate groups,
R⁹ stands for hydrogen, C₁-C₁₈ alkyl that is optionally substituted in one or more places with carboxy, phosphoryl or sulfonate groups, or for an aryl group with one or more heteroatoms that is optionally substituted with aralkyl or sulfonate,
R¹⁰ and R¹¹ are the same or different and mean hydrogen or C₁-C₁₈ alkyl, aryl, heteroaryl or acyl that is optionally substituted with hydroxy and/or amino; or C₁-C₁₈ alkyl, C₃-C₈ cycloalkyl or C₃-C₈ cycloalkenyl that is optionally interrupted by one or more oxygen atoms,
or
R¹⁰ and R¹¹ together with the nitrogen atom of the amino group forms a C₃-C₈ cycloalkyl, which can contain one or more additional heteroatoms,
M stands for hydrogen, C₁-C₁₈ alkyl that is optionally substituted by one or more hydroxy groups and/or amino groups, or aryl or heteroaryl that is optionally substituted in one or more places with halogen, C₁-C₆ alkyl or C₁-C₆ alkoxy,
n is 0, 1 or 2
and
at least one of radicals R¹, R², R⁴, and R⁵ is substituted with an —S(O)ₙR⁶ group, as well as optical isomers and salts thereof.

2. Compounds of general formula I, according to claim 1, in which
R¹ stands for group —S(O)ₙR⁶₁
R² stands for hydrogen,
R³ stands for oxygen or group ═NOR⁷,
R⁴ and R⁵ stand for hydrogen, halogen, hydroxy, nitroso, nitro, C₁-C₁₀ alkoxy that is optionally interrupted by one or more oxygen atoms; C₁-C₁₈ alkyl that is optionally substituted in one or more places with halogen, hydroxy and/or amino; aryl, benzyl, benzyloxy or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —NHCO-C₁-C₆ alkyl group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,
and
R⁶ stands for hydrogen or C₁-C₁₈ alkyl,
or
R¹ and R²
or
R⁴ and R⁵, independently of one another, form a ring with 1 to 4 —CH₂ groups, which, independently of one another, optionally are substituted in one or two places with halogen, hydroxy, nitroso, nitro, C₁-C₁₀ alkoxy; C₁-C₁₈ alkyl that is optionally substituted in one or more places with halogen, hydroxy and/or amino; aryl or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, —CH₂COOM group, —CONR¹⁰R¹¹ group, —NR¹⁰R¹¹ group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,
R⁷ stands for hydrogen, C₂-C₁₈ alkenyl, or C₁-C₁₈ alkyl,
R⁸ stands for aryl that is optionally substituted by one or more carboxyl, phosphoryl or sulfonate groups,
R¹⁰ and R¹¹ are the same or different and mean hydrogen or C₁-C₁₈ alkyl, aryl, heteroaryl or acyl that is optionally substituted with hydroxy and/or amino; or C₁-C₁₈ alkyl, C₃-C₈ cycloalkyl or C₃-C₈ cycloalkenyl that is optionally interrupted by one or more oxygen atoms,
or
R¹⁰ or R¹¹ together with the nitrogen atom of the amino group forms a C₃-C₈ cycloalkyl, which can include one or more additional heteroatoms,
M stands for hydrogen, C₁-C₁₈ alkyl that is optionally substituted by one or more hydroxy groups and/or amino groups; or aryl or heteroaryl that is optionally substituted in one or more places with halogen, C₁-C₆ alkyl or C₁-C₆ alkoxy,
and
n is 0, 1 or 2, as well as optical isomers and salts thereof.

3. Compounds of general formula I, according to claims 1 to 2, in which
R¹ stands for group —S(O)ₙR⁶,
R² stands for hydrogen,
R³ stands for oxygen or group ═NOR⁷,
R⁴ and R⁵ stand for hydrogen, halogen, hydroxy, nitroso, nitro, C₁-C₁₀ alkoxy that is optionally interrupted by one or more oxygen atoms; C₁-C₁₈ alkyl that is optionally substituted in one or more places with halogen, hydroxy and/or amino; aryl, benzyl, benzyloxy or heteroaryl that is optionally substituted in one or more places with halogen, C₁-C₆ alkyl, hydroxy, amino and/or C₁-C₆ alkoxy; or aralkyl, aryloxy, methylenaryloxy, C₃-C₈ cycloalkyl, C₃-C₈ cycloalkenyl or C₃-C₇ methylenecycloalkyl that is optionally substituted in one or more places with halogen, C₁-C₆ alkyl, hydroxy, amino and/or C₁-C₆ alkoxy and that optionally contains one or more heteroatoms; or hydroxylamino, phosphate, phosphonate, sulfate, sulfonate, sulfonamide that is optionally substituted in one or more places with halogen, C₁-C₆ alkyl, hydroxy, amino and/or C₁-C₆ alkoxy; or a —COM group, —COOM group, −CH₂COOM group, —CONR¹⁰R¹¹ group, —NR¹⁰R¹¹ group, —NHCO-C₁-C₆ alkyl group, —SO₂NR¹⁰R¹¹ group, —N═N—R⁸ group or an —S(O)ₙR⁶ group; or an O-glycoside or N-glycoside, whereby the glycosides are selected from the group of mono- or disaccharides,
and
R⁶ stands for C₁-C₆ alkyl,
R⁷ stands for hydrogen or C₂-C₆ alkenyl,
R⁸ stands for aryl that is optionally substituted by one or more carboxyl, phosphoryl or sulfonate groups,
R¹⁰ and R¹¹ are the same or different and mean hydrogen or C₁-C₁₈ alkyl, aryl, heteroaryl or acyl that is optionally substituted with hydroxy and/or amino; or C₁-C₁₈ alkyl, C₃-C₈ cycloalkyl or C₃-C₈ cycloalkenyl that is optionally interrupted by one or more oxygen atoms,
or
R¹⁰ and R¹¹ together with the nitrogen atom of the amino group forms a C₃-C₈ cycloalkyl, which can contain one or more additional heteratoms,
M stands for hydrogen, C₁-C₁₈ alkyl that is optionally substituted by one or more hydroxy groups and/or amino groups, or aryl or heteroaryl that is optionally substituted in one or more places with halogen, C₁-C₆ alkyl or C₁-C₆ alkoxy,
and
n is 0, 1 or 2, as well as optical isomers and salts thereof.

4. Compounds of general formula I, according to claims 1 to 3, in which
R¹ stands for the group S(O)ₙR⁶,
R² stands for hydrogen,
R³ stands for oxygen or the group —NOR⁷,
R⁴ stands for hydrogen, benzyloxy or for an —NHCOCH₃ group,
R⁵ stands for hydrogen,
R⁶ stands for C₁-C₆ alkyl,
R⁷ stands for hydrogen or C₂-C₆ alkenyl,
and
n is 0, 1 or 2, as well as optical isomers and salts thereof.

5. Use of the compounds of general formula I, according to claims 1 to 4, for the production of a pharmaceutical agent for treating cancer, auto-immune diseases, chemotherapy agent-induced alopecia and mucositis, cardiovascular diseases, infectious diseases, nephrological diseases, chronic and acute neurodegenerative diseases and viral infections.

6. Use according to claim 5, characterized in that cancer is defined as solid tumors and leukemia; auto-immune diseases are defined as psoriasis, alopecia and multiple sclerosis; cardiovascular diseases are defined as stenoses, arterioscleroses and restenoses; infectious diseases are defined as diseases that are caused by unicellular parasites; nephrological diseases are defined as glomerulonephritis; chronic neurodegenerative diseases are defined as Huntington's disease, amyotrophic lateral sclerosis, Parkinson's disease, AIDS dementia and Alzheimer's disease; acute neurodegenerative diseases are defined as ischemias of the brain and neurotraumas; and viral infections are defined as cytomegalic infections, herpes, hepatitis B or C, and HIV diseases.

7. Pharmaceutical agents that contain at least one compound according to claims 1 to 4.

8. Pharmaceutical agents according to claim 7 for treating cancer, auto-immune diseases, cardiovascular diseases, infectious diseases, nephrological diseases, neurodegenerative diseases and viral infections.

9. Compounds according to claims 1 to 4 and pharmaceutical agents according to claims 7 to 8 with suitable formulation substances and vehicles.

10. Use of the compounds of general formula I, according to claims 1 to 4, as inhibitors of cyclin-dependent kinases.

11. Use according to claim 10, wherein the kinase is CDK1, CDK2, CDK3, CDK4, CDK5, CDK6, CDK7, CDK8 or CDK9.

12. Use of the compounds of general formula I, according to claims 1 to 4, as inhibitors of the glycogen-synthase-kinase (GSK-3β).

13. Use of the compounds of general formula I, according to claims 1 to 4, in the form of a pharmaceutical preparation for enteral, parenteral and oral administration.
